(19) United States
(12) Patent Application Publication
(10) Pub. No.: US 2022/0409776 A1
(43) Pub. Date: Dec. 29, 2022

(54) A MEDICAL TUBING COMPRISING THERMOPLASTIC POLYURETHANE

(71) Applicant: BASF SE, Ludwigshafen am Rhein (DE)

(72) Inventors: Felicia Bokel, Wyandotte, MI (US); Mark D. Swartzlander, Wyandotte, MI (US); Agnieszka D. Gajewski, Wyandotte, MI (US)

(73) Assignee: BASF SE, Ludwigshafen am Rhein (DE)

(21) Appl. No.: 17/756,722

(22) PCT Filed: Dec. 1, 2020
(86) PCT No.: PCT/EP2020/084013

§ 371 Date: Jun. 1, 2022

Related U.S. Application Data
(60) Provisional application No. 62/942,831, filed on Dec. 3, 2019.

(30) Foreign Application Priority Data
Jan. 7, 2020 (EP) 20150453.7

(51) Int. Cl. A61L 29/06; C08G 18/32

(52) U.S. Cl. CPC A61L 29/06; C08G 18/3206; C08G 18/4213

(57) ABSTRACT

A medical tubing contains a thermoplastic polyurethane.

### FIELD OF INVENTION

The present invention relates to a medical tubing comprising a thermoplastic polyurethane.

### BACKGROUND OF THE INVENTION

Traditional TPUs are known to have advantageous properties, which render it useful for a wide range of applications, can be varied by modifying the ingredients. Viscoelasticity in TPUs, among other properties, is usually temperature sensitive and is maximised, when the polymer undergoes a glass transition. By manipulating the structure and composition of the soft segment phase so that the glass transition temperature (Tg) approximately coincides with the use temperature of the material, the viscoelastic nature of the material can be maximised. Further, since the TPUs are segmental polyurethanes, i.e. they separate to form a microstructure of hard and soft domains, their properties can be controlled by optimizing these segments. Generally, the hard segment in the TPU is made of chain extenders and isocyanates, while the soft segment comprises polyols. A right balance between the TPU structure and characteristics provides for advantageous properties that can be utilized in different applications. For instance, the application of viscoelastic TPU materials in the medical field, particularly for medical tubings, is one of the most sought for.

As a requirement of a medical tubing application, the TPU material ought to have optimum viscoelastic properties. To name a few, hard segment, Tg and shore hardness, are important for determining the applicability of a TPU material for a medical tubing application. Many attempts have been made to strike the right balance of these properties or at least one or more of them. However, none of them were directed for medical tubing applications.

U.S. Pat. No. 5,574,092 A discloses a rigid TPU having a Tg of at least 50° C. and comprising a hard segment based on a diisocyanate and a chain extender mixture comprising an aromatic diol. According to the examples, very brittle materials having an elongation at break of less than 170% are obtained.

U.S. Pat. No. 5,627,254 A also discloses rigid TPU comprising units of butanediol (BDO) and a polyethylene glycol (PEG) of the HO—(CH₂CH₂O)ₙ—H type, where n is an integer from 2 to 6. These materials have the disadvantage of being brittle and difficult to process.

WO 2015/063062 A1 relates to TPUs obtainable or obtained by reacting at least one aliphatic polyisocyanate, at least one chain extender and at least one polyol composition, wherein the polyol composition comprises a polyol selected from the group consisting of polyetherols and at least one bisphenol derivative selected from the group consisting of bisphenol A derivatives having a weight average molecular weight Mw>315 g/mol and bisphenol S derivatives having a weight average molecular weight Mw>315 g/mol, wherein at least one of the OH groups of the bisphenol derivative has been alkoxylated, and to processes for producing such TPUs and to the use of a TPU of the invention for production of extrusion products, films and shaped bodies. Such aliphatic TPUs having a hardness of >70 Shore D have a low modulus of elasticity and only inadequate elongation at break. A further disadvantage is the use of bisphenol A, which is of some toxicological concern.

Typically, hard TPUs that are obtained by reaction of isocyanates and chain extenders, for example hexane-1,6-diol or cyclohexane-1,4-dimethanol, have a hard segment content of not less than 90%. These materials have a high hardness and a high dimensional stability but are very brittle and only have an elongation at break of less than 200% or even less than 100%.

Other TPU materials known in the state of the art, have a Tg ranging between −50° C. to −25° C. and do not exhibit a strong viscoelastic response at room temperature. The viscoelastic response is often characterized as “tired” or “dead” and can visually be identified by a slow recovery time after deformation. To achieve this viscoelastic behaviour, often mechanical properties such as tensile strength, yield strength and elongation at break are sacrificed. Tg modification, particularly the fraction of hard segment in the TPU, can be carried out by varying the monomers. However, there are always limitations in doing so, for example, TDI results in sticky material, MDI would give a low Tg, high molecular weight chain extenders and/or polyols would negatively impact the hard segment fraction, etc.

These TPU materials are known for producing shaped bodies, for example, laminated systems, coatings for sports equipment or floor coatings, consumer articles or housings for domestic articles such as toothbrushes, razors housings, displays, spectacle frames or spectacle lenses, parts of computers or telephones, plugs, parts of automobile interior fit out, and footwear parts such as caps for safety footwear. Thus, a medical tubing application comprising viscoelastic TPU materials is not known in the state of the art.

It was, therefore, an object of the presently claimed invention to provide a medical tubing comprising a TPU material, said material showcasing viscoelastic behaviour, being transparent and having acceptable mechanical properties such as, but not limited to, tensile strength, elongation at break, and tear strength which can be produced in a simple and inexpensive manner.

### SUMMARY OF THE INVENTION

Surprisingly, it has been found that the above identified object is met by providing a medical tubing comprising a viscoelastic TPU having a shore hardness of less than 95 A, determined according to DIN ISO 7619.

Accordingly, in one aspect, the presently claimed invention is directed to a medical tubing comprising a TPU obtained by reacting:

(a) a polyisocyanate composition,

(b) at least one chain extender, and

(c) at least one polyol composition,

wherein the polyol composition comprises at least one polyester polyol (P1) which has a weight average molecular weight Mw in the range of 500 g/mol to 3000 g/mol and has at least one aromatic polyester block (B1), wherein the aromatic polyester block (B1) is present in between 20 wt. % to 80 wt. %, based on the total weight of the polyester polyol (P1), wherein the TPU has a shore hardness of less than 95 A, determined according to DIN ISO 7619.

In another aspect, the presently claimed invention is directed to a process for preparing the above medical tubing.

### DETAILED DESCRIPTION OF THE INVENTION

Before the present compositions and formulations of the invention are described, it is to be understood that this invention is not limited to particular compositions and formulations described, since such compositions and formulation may, of course, vary. It is also to be understood that the terminology used herein is not intended to be limiting, since the scope of the present invention will be limited only by the appended claims.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(A)”, “(B)” and “(C)” or “(a)”, “(b)”, “(c)”, “(d)”, “i”, “ii” etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment but may. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art. For example, in the appended claims, any of the claimed embodiments can be used in any combination.

Furthermore, the ranges defined throughout the specification include the end values as well, i.e. a range of 1 to 10 implies that both 1 and 10 are included in the range. For the avoidance of doubt, the applicant shall be entitled to any equivalents according to applicable law.

Medical Tubing

An aspect of the present invention is embodiment 1, directed to a medical tubing comprising a TPU obtained by reacting:

(a) a polyisocyanate composition,

(b) at least one chain extender, and

(c) at least one polyol composition,

wherein the polyol composition comprises at least one polyester polyol (P1) which has a weight average molecular weight Mw in the range of 500 g/mol to 3000 g/mol and has at least one aromatic polyester block (B1), wherein the aromatic polyester block (B1) is present in between 20 wt. % to 80 wt. %, based on the total weight of the polyester polyol (P1), wherein the TPU has a shore hardness of less than 95 A, determined according to DIN ISO 7619.

Thermoplastic Polyurethane (TPU)

In one embodiment, the TPU in the embodiment 1 is obtained by reacting:

(a) polyisocyanate composition,

(b) at least one chain extender, and

(c) at least one polyol composition,

wherein the polyol composition comprises at least one polyester polyol (P1) which has a weight average molecular weight Mw in the range of 500 g/mol to 3000 g/mol and has at least one aromatic polyester block (B1), wherein the aromatic polyester block (B1) is present in between 20 wt. % to 80 wt. %, based on the total weight of the polyester polyol (P1), wherein the TPU has a shore hardness of less than 95 A, determined according to DIN ISO 7619.

In one embodiment, the viscoelastic behaviour in the TPU in the embodiment 1 is determined by the shore hardness value, glass transition temperature Tg and hard segment content. Accordingly, in the present context, the TPU material is considered viscoelastic and, therefore, suitable for medical tubing, if (i) the shore hardness is less than 95 A, (ii) the Tg ranges between −5° C. to 30° C. and (iii) the hard segment content is less than 50%.

In an embodiment, the TPU in the embodiment 1 has a shore A hardness ranging between 30 to 95, determined according to DIN ISO 7619. In still another embodiment, it is in between 50 to 95, or in between 70 to 95.

In another embodiment, the TPU in the embodiment 1 has a glass transition temperature Tg ranging between −5° C. to 30° C. The Tg value is determined by dynamic mechanical analysis according to DIN EN ISO 6721 at temperatures ranging between −80° C. to 140° C. with increments of 5° C. and at a frequency of 1 Hz in torsion mode. Dynamic mechanical analysis (DMA) or dynamic mechanical thermal analysis (DMTA) yields information about the mechanical properties of a specimen placed in minor, usually sinusoidal, oscillation of a function of time and temperature by subjecting it to a small, usually sinusoidal, oscillating force. In order to measure the Tg value of the TPU, storage modulus (G′) and loss modulus (G″) are first determined. The storage modulus (G′) represents the stiffness of the polymer material and is proportional to the energy stored during a loading cycle. The loss modulus (G″) is defined as being proportional to me energy dissipated during one loading cycle. It represents, for example, the energy lost as heat, and is a measure of vibrational energy that has been converted during vibration and that cannot be recovered. Next, phase angle delta (δ) is measured, which is the phase difference between dynamic stress and dynamic strain in the TPU subjected to a sinusoidal oscillation. Loss factor tan delta is the ratio of loss modulus (G′) to storage modulus (G″). It is a measure of the energy lost, expressed in terms of the recoverable energy, and represents mechanical damping or internal friction in the TPU. A high tan delta value is indicative of a material that has a high, non-elastic strain component, while a low value indicates one that is more elastic. Often, the Tg value is taken to be the temperature of the maximum loss modulus (G″ₘₐₓ) or the maximum loss factor (max tan delta), as shown in the examples described below.

In one embodiment, the TPU in the embodiment 1 has a hard segment content of less than 50%. The hard segment content of the TPU is defined by the formula:

$$Hard segment fraction = \{ {\sum }_{x = 1}^{k} [ ( {m}_{KV , CE} / {M}_{KV , CE} ) * {M}_{Iso} + {m}_{KV , CE} ] \} / {m}_{total}$$

wherein,

m_{KV,CE} is the mass of the at least one chain extender in g,

M_{KV,CE} is the molar mass of the at least one chain extender in g/mol,

M_{Iso} is the molar mass of the polyisocyanate in g/mol,

mₜₒₜₐₗ is the total mass of all the starting materials in g,

k is the number of chain extenders.

In another embodiment, the hard segment of the TPU in the embodiment 1 is in between 10% to 45%, or in between 20% to 45%.

Primarily, the viscoelastic properties in the TPU material are governed by the choice and amount of the ingredients, such as but not limited to, (a) the polyisocyanate composition, (b) at least one chain extender, and (c) at least one polyol composition. However, other ingredients, for instance, conventional additives and/or cross-linking agents may be added to impart additional properties and/or aid in processing of the TPU material.

Polyisocyanate Composition (a)

In one embodiment, the polyisocyanate composition in the embodiment 1 comprises an aliphatic polyisocyanate, or an aromatic polyisocyanate, or a mixture thereof. Aromatic polyisocyanates include those in which two or more of the isocyanato groups are attached directly and/or indirectly to the aromatic ring. Further, it is to be understood here that the polyisocyanate includes both monomeric and polymeric forms of the aliphatic or aromatic polyisocyanates. By the term “polymeric”, it is referred to the polymeric grade of the aliphatic or aromatic polyisocyanate comprising different oligomers and homologues.

Suitable aliphatic polyisocyanates can be selected from tetramethylene 1,4-diisocyanate, pentamethylene 1,5-diisocyanate, hexamethylene 1,6-diisocyanate, decamethylene diisocyanate, 1,12-dodecane diisocyanate, 2,2,4-trimethyl-hexamethylene diisocyanate, 2,4,4-trimethylhexamethylene diisocyanate, 2-methyl-1,5-pentamethylene diisocyanate, cyclobutane-1,3-diisocyanate, 1,2-, 1,3- and 1,4-cyclohexane diisocyanates, 2,4- and 2,6-methylcyclohexane diisocyanate, 4,4′- and 2,4′-dicyclohexyldiisocyanates, 1,3,5-cyclohexane triisocyanates, isocyanatomethylcyclohexane isocyanates, isocyanatoethylcyclohexane isocyanates, bis(isocyanatomethyl)-cyclohexane diisocyanates, 4,4′-diisocyanatodicyclohexylmethane, pentamethylene 1,5-diisocyanate, isophorone diisocyanate and mixtures thereof.

In another embodiment, the polyisocyanate composition in the embodiment 1 comprises aromatic polyisocyanate. Suitable aromatic polyisocyanate is selected from toluene diisocyanate; polymeric toluene diisocyanate, methylene diphenyl diisocyanate and/or polymeric methylene diphenyl diisocyanate; m-phenylene diisocyanate; 1,5-naphthalene diisocyanate; 4-chloro-1; 3-phenylene diisocyanate; 2,4,6-toluylene triisocyanate, 1,3-diisopropylphenylene-2,4-diisocyanate; 1-methyl-3,5-diethylphenylene-2,4-diisocyanate; 1,3,5-triethylphenylene-2,4-diisocyanate; 1,3,5-triisoproply-phenylene-2,4-diisocyanate; 3,3′-diethyl-bisphenyl-4,4′-diisocyanate; 3,5,3′,5′-tetraethyl-diphenylmethane-4,4′-diisocyanate; 3,5,3′,5′-tetraisopropyldiphenylmethane-4,4′-diisocyanate; 1-ethyl-4-ethoxy-phenyl-2,5-diisocyanate; 1,3,5-triethyl benzene-2,4,6-triisocyanate; 1 ethyl-3,5-diisopropyl ben-zene-2,4,6-triisocyanate, tolidine diisocyanate, and 1,3,5-triisopropyl benzene-2,4,6-triisocyanate. It is to be understood here that all isomeric corms or the poiyisocyanates are within the context of this invention. For example, toluene diisocyanate (TDI) includes 2,4-TDI and 2,6-TDI, or methylene diphenyl diisocyanate (MDI) includes 2,2′-MDI, 2,4′-MDI and 4,4′-MDI. A mixture of these isomeric forms is also to be considered within the context of this invention.

In yet another embodiment, the aromatic polyisocyanate is selected from toluene diisocyanate; polymeric toluene diisocyanate, methylene diphenyl diisocyanate and/or polymeric methylene diphenyl diisocyanate; m-phenylene diisocyanate; 1,5-naphthalene diisocyanate; 4-chloro-1; 3-phenylene diisocyanate; 2,4,6-toluylene triisocyanate, 1,3-diisopropylphenylene-2,4-diisocyanate; 1-methyl-3,5-diethylphenylene-2,4-diisocyanate; 1,3,5-triethylphenylene-2,4-diisocyanate; 1,3,5-triisoproply-phenylene-2,4-diisocyanate; 3,3′-diethyl-bisphenyl-4,4′-diisocyanate; 3,5,3′,5′-tetraethyl-diphenylmethane-4,4′-diisocyanate; 3,5,3′,5′-tetraisopropyldiphenylmethane-4,4′-diisocyanate, and 1-ethyl-4-ethoxy-phenyl-2,5-diisocyanate.

In still another embodiment, it is selected from toluene diisocyanate; polymeric toluene diisocyanate, methylene diphenyl diisocyanate and/or polymeric methylene diphenyl diisocyanate; m-phenylene diisocyanate, and 1,5-naphthalene diisocyanate. In a further embodiment, the aromatic polyisocyanate is methylene diphenyl diisocyanate and/or polymeric methylene diphenyl diisocyanate.

In still another embodiment, the polyisocyanate composition does not include TDI.

MDI is available in three different isomeric forms, namely 2,2′-MDI, 2,4′-MDI and 4,4′-MDI. Polymeric MDI includes oligomeric species and MDI isomers. Thus, polymeric MDI may contain a single MDI isomer or isomer mixtures of two or three MDI isomers, the balance being oligomeric species. Polymeric MDI tends to have isocyanate functionalities of higher than 2.0. The isomeric ratio as well as the amount of oligomeric species can vary in wide ranges in these products. For instance, polymeric MDI may typically contain 30 wt.-% to 80 wt.-% of MDI isomers, the balance being said oligomeric species. The MDI isomers are often a mixture of 4,4′-MDI, 2,4′-MDI and very low levels of 2,2′-MDI.

In one embodiment, the polyisocyanate composition may optionally comprise prepolymers, wherein some of the OH components have been reacted with an isocyanate in a preceding reaction step. These prepolymers are reacted with the remaining OH components in a further step, the actual polymer reaction, and then form the TPU. The use of prepolymers makes it possible also to use OH components having secondary alcohol groups.

In another embodiment, the polyisocyanate composition in the embodiment 1 may further comprise at least one solvent. Suitable solvents are known to those skilled in the art. For example, nonreactive solvents such as ethyl acetate, methyl ethyl ketone, tetrahydrofuran and hydrocarbons may be used.

In one embodiment, the polyisocyanate composition in the embodiment 1 is added in an amount suitable to yield the TPU with a hard segment content of less than 50%, or in between 10% to 45%, or in between 20% to 45%, as disclosed herein. However, in another embodiment, the polyisocyanate composition in the embodiment 1 is present in an amount in between 10 wt. % to 70 wt. %, based on the total weight of the TPU. In yet another embodiment, the amount is in between 15 wt. % to 70 wt. %, or in between 15 wt. % to 65 wt. %, or in between 20 wt. % to 65 wt. %. In still another embodiment, it is in between 20 wt. % to 60 wt. %, or in between 24 wt. % to 60 wt. %, or in between 24 wt. % to 55 wt. %. In a further embodiment, it is present in between 24 wt. % to 52 wt. %.

Chain Extender (b)

In one embodiment, chain extenders in the embodiment 1 are compounds having hydroxyl or amino groups, especially having 2 hydroxyl or amino groups. According to the invention, however, it is possible that mixtures of different compounds are used as chain extenders. In another embodiment, suitable chain extenders in the embodiment 1 are compounds having hydroxyl groups, especially diols.

In another embodiment, the chain extenders in the embodiment 1 can be selected from aliphatic, araliphatic, aromatic and cycloaliphatic diols having a molecular weight in between 60 g/mol to 300 g/mol, or in between 50 g/mol or 220 g/mol. In yet another embodiment, chain extenders in the embodiment 1 comprise of alkanediols having 2 to 10 carbon atoms in the alkylene radical, especially di-, tri-, tetra-, penta-, hexa-, hepta-, octa-, nona- and/or deca-alkylene glycols.

In still another embodiment, chain extenders in the embodiment 1 can be selected from 1,2-ethylene glycol, diethylene glycol, propane-1,3-diol, butane-1,4-diol, hexane-1,6-diol, and cyclohexane-1,4-dimethanol.

In one embodiment, it is possible that more than one chain extender is used in the embodiment 1. However, at least one of the chain extenders should have a molecular weight ranging between 60 g/mol to 300 g/mol. The second or further chain extender may, if required, have a molecular weight of more than 300 g/mol.

In another embodiment, the molar ratio between the at least one chain extender (b) and the at least one polyester polyol (P1) in the embodiment 1 is in between 1.0:5.0 to 5.0:1.0. In another embodiment, the ratio is in between 1.0:4.0 to 5.0:1.0, or in between 1.0:3.0 to 5.0:1.0, or in between 1.0:2.0 to 5.0:1.0. In yet another embodiment, it is in between 1.0:1.0 to 5.0:1.0, or in between 2.0:1.0 to 5.0:1.0.

Polyol Composition (c)

In one embodiment, the polyol composition in the embodiment 1 comprises at least one polyester polyol (P1) which has a weight average molecular weight Mw in the range from 500 g/mol to 3000 g/mol. In addition, the polyester polyol (P1) has an aromatic polyester block (B1), wherein the polyester polyol (P1) includes 20% to 70% by weight of the aromatic polyester block (B1), based on the overall polyester polyol (P1). In the context of the present invention, this is understood to mean that the aromatic polyester block (B1) may be a polyester of an aromatic dicarboxylic acid and an aliphatic diol or a polyester of an aliphatic dicarboxylic acid and an aromatic diol. In another embodiment, the aromatic polyester block (B1) in the context of the present invention is a polyester of an aromatic dicarboxylic acid and an aliphatic diol. Suitable aromatic dicarboxylic acids are, for example, terephthalic acid, isophthalic acid or phthalic acid, preferably terephthalic acid. Accordingly, suitable polyester polyols (P1) in the context or me present invention are those that have, for example, at least one polyethylene terephthalate block or at least one polybutylene terephthalate block, where the number of repeat units in the aromatic systems is at least 2 in series. Preferably, the aromatic polyester block (B1) is obtained in the reaction by a degradation reaction of a higher molecular weight aromatic polyester, where the higher molecular weight aromatic polyester is typically prepared in a separate step prior to the conversion to polyester polyol (P1) in order to ensure a sufficient block length of the repeat units of the aromatic system.

In a further embodiment, the aromatic polyester block (B1) in the embodiment 1 is a polyester of an aromatic dicarboxylic acid and an aliphatic diol. In another embodiment, the aromatic polyester block (B1) is a polyethylene terephthalate block or a polybutylene terephthalate block. In still another embodiment, the aromatic polyester block (B1) is a polyethylene terephthalate block.

In another embodiment, the polyester polyol (P1) in the embodiment 1 is based on aromatic polyesters, such as polybutylene terephthalate (PBT) or polyethylene terephthalate block (PET). Such polyester polyols (P1) are prepared by reacting the aromatic polyester with dicarboxylic acids and diols to give mixed aromatic/aliphatic polyester diols. For example, it is possible in the context of the present invention to react the aromatic polyester in solid or liquid form with dicarboxylic acids and diols. In one embodiment, the aromatic polyester used typically has a higher molecular weight than the blocks (B1) present in the polyester polyol (P1).

Polyethylene terephthalate (PET) is a thermoplastic polymer prepared by polycondensation. The quality of the PET, and its physical properties such as toughness or durability, are dependent on the chain length. Older PET synthesis methods are based on the transesterification of dimethyl terephthalate with ethylene glycol. Nowadays, PET is synthesized almost exclusively by direct esterification of terephthalic acid with ethylene glycol. In the same way, terephthalic acid can also be reacted with butane-1,4-diol to give polybutylene terephthalate (PBT). This likewise thermoplastic polymer is available under brands such as CRASTIN® (DuPont), POCAN® (Lanxess), ULTRADUR® (BASF) or ENDURAN® and VESTODUR® (SABIC IP). Its chemical and physical/technical properties correspond largely to those of PET.

In one embodiment, the aromatic polyester block (B1) in the embodiment 1 is present in between 20 wt. % to 70 wt. %, based on the total weight of the polyester polyol (P1). In another embodiment, it is present in between 25 wt. % to 65 wt. %, or in between 25 wt. % to 60 wt. %, or in between 25 wt. % to 55 wt. %. In yet another embodiment, it is present in between 25 wt. % to 55 wt. %, or in between 25 wt. % to 50 wt. %.

In another embodiment, the polyester polyol (P1) in the embodiment 1 has a weight average molecular weight Mw in the range of 500 g/mol to 3000 g/mol, or in between 500 g/mol to 2700 g/mol, or in between 500 g/mol to 2500 g/mol. In yet another embodiment, it is in between 500 g/mol to 2300 g/mol, or in between 700 g/mol to 2300 g/mol, or in between 900 g/mol to 2300 g/mol. The weight average molecular weight Mw is calculated using the following formula, where z is the functionality of the polyester polyol (P1) and z ranges between 1.98 to 2.02:

$${M}_{w} = 1000 ( \frac{mg}{g} ) \times \frac{z \times 56.106 ( \frac{g}{mol} )}{OHN ( \frac{mg}{g} )}$$

In one embodiment, the polyester polyol (P1) in the embodiment 1 has a hydroxyl number ranging between 35 mg KOH/g to 250 mg KOH/g, determined according to DIN 53240. In another embodiment, it is in the range between 35 mg KOH/g to 200 mg KOH/g, or in between 35 mg KOH/g to 150 mg KOH/g, or in between 35 mg KOH/g to 100 mg KOH/g. In yet another embodiment, it is in the range between 40 mg KOH/g to 100 mg KOH/g, or in between 40 mg KOH/g to 70 mg KOH/g, or in between 45 mg KOH/g to 60 mg KOH/g.

In one embodiment, it is also possible to use aromatic polyesters such as polybutylene terephthalate (PBT) or polyethylene terephthalate (PET) that are obtained from recycling processes. For example, polyethylene terephthalate can be used in the form of flakes or as pellets that are obtained from plastic recycling processes. Materials of this kind typically have molecular weights of about 12,000 g/mol.

In another embodiment, suitable polyester polyols (P1) in the embodiment 1 can also be obtained using aromatic polyesters such as polybutylene terephthalate or polyethylene terephthalate with higher molecular weight and diols by transesterification. Suitable reaction conditions are known per se to those skilled in the art.

In still another embodiment, in the preparation of the polyester polyols (P1) in the embodiment 1, diols having 2 to 36 carbon atoms, for example ethanediol, propanediol, butanediol, pentanediol, hexanediol, diethylene glycol, triethylene glycol, or else diols that are obtained from dimerized fatty acids, can be used. In a further embodiment, it is also possible to use mixtures of two or more of these diols. For example, butane-1,4-diol or mixtures comprising butane-1,4-diol can be used. It is also possible to use short polyether diols, for example PTHF 250 or PTHF 650 or a short-chain polypropylene glycol such as a PPG 500. Dicarboxylic acids used may, for example, be linear or branched-chain diacids having four to 36 carbon atoms or mixtures thereof. For example, it is also possible to use dimerized fatty acids. For instance, adipic acid, succinic acid, glutaric acid or sebacic acid or a mixture of these acids can be used. Of particular relevance is adipic acid. According to the invention, in the preparation of the polyester polyols (P1) in the embodiment 1, it is also possible to use further polyester diols as feedstocks, for example butanediol adipate or ethylene adipate.

In another embodiment, the polyol composition in the embodiment 1 comprises a further polyol (P2) selected from the group consisting of polyetherols, polyesterols, polycarbonate alcohols and hybrid polyols. Higher molecular weight compounds having hydrogen atoms reactive toward isocyanates that are used may be the commonly known polyols having compounds reactive toward isocyanates.

Polyols (P2) are fundamentally known to those skilled in the art and described for example in “Kunststoffhandbuch, Band 7, Polyurethane” [Plastics Handbook, volume 7, Polyurethanes], Carl Hanser Verlag, 3rd edition 1993, chapter 3.1. Copolymers may also be used in the context of the present invention. The number-average molecular weight of polyols used in accordance with the invention can be in between 0.5×10³ g/mol to 8×10³ g/mol, or in between 0.6×10³ g/mol to 5×10³ g/mol, or in between 0.8×10³ g/mol and 3×10³ gi/mol. These polyols nave an average functionality with respect to isocyanates of 1.8 to 2.3, or in between 1.9 to 2.2.

Polyesterols used may be polyesterols based on diacids and diols. Diols used are diols having 2 to 10 carbon atoms, for example ethanediol, propanediol, butanediol, pentanediol, hexanediol or di- or triethylene glycol, especially butane-1,4-diol or mixtures thereof. Diacids used may be any known diacids, for example linear or branched-chain diacids having four to 12 carbon atoms or mixtures thereof. Adipic acid may be used as a suitable diacid.

Polyetherols used may be, such as but not limited to, polyethylene glycols, polypropylene glycols and polytetrahydrofurans.

In one embodiment, the polyol (P2) is a polytetrahydrofuran (PTHF) having a weight average molecular weight Mw in the range of 600 g/mol to 3000 g/mol. According to the invention, as well as PTHF, various other polyethers are suitable, nevertheless polyesters, block copolymers and hybrid polyols, for example poly(ester/amide), can also be used.

In one embodiment, the polyol (P2) in the polyol composition in the embodiment 1 has an average functionality between 1.8 and 2.3, or in between 1.9 and 2.2, or 2.0. In another embodiment, the polyols (P2) used in accordance with the invention have solely primary hydroxyl groups.

According to the invention, the polyol (P2) may be used in pure form or in the form of a composition comprising the polyol (P2) and at least one solvent. Suitable solvents are known per se to the person skilled in the art.

The additional polyol (P2) can be used in a molar ratio ranging between 10.0:1.0 to 1.0:10.0 relative to the polyester polyol (P1). In another embodiment, the molar ratio is in between 9.0:1.0 to 1.0:9.0, or in between 5.0:1.0 to 1.0:5.0.

In one embodiment, the polyol composition in the embodiment 1 is present in an amount in between 50 wt. % to 90 wt. %, based on the total weight of the TPU. In another embodiment, it is present in between 50 wt. % to 85 wt. %, or in between 50 wt. % to 80 wt. %. In still another embodiment, it is present in between 50 wt. % to 75 wt. %.

Cross-Linking Agent

As the name suggests, the cross-linking agents react with the TPU material, as described herein, to form cross-links, i.e., to form cross-linked TPU. The cross-linking agent reacts with the TPU to create a reinforced polymer network. The cross-linking agent comprises a TPU carrier, which is different than the TPU described herein, and an isocyanate component. The crosslinking agent typically includes 60 parts by weight of the TPU carrier and typically less than 48 parts by weight of the isocyanate component, based on 100 parts by weight of the cross-linking agent.

The isocyanate component of the cross-linking agent includes at least one isocyanate. Isocyanates suitable for use in the isocyanate component include, but are not limited to, aliphatic and aromatic isocyanates, as described herein. The isocyanate component of the cross-linking agent may include an isocyanate prepolymer. The isocyanate prepolymer is typically a reaction product of an isocyanate and a polyol and/or a polyamine.

In an embodiment, the cross-linking agent comprises the TPU carrier and the isocyanate component comprises the isocyanate prepolymer, 4,4′-MDI, and MDI mixed isomers. In this embodiment, the cross-linking agent includes less than 60 parts by weight of the TPU carrier, less than 25 parts by weight of the isocyanate prepolymer, 20 parts by weight MDI, and less than 3 parts by weight MDI mixed isomers, based on 100 parts by weight of the cross-linking agent.

When the TPU material in the embodiment 1 comprises the cross-linking agent, it is present in an amount in between 0.1 wt. % to 10 wt. %, based on the total weight of the TPU.

Further Ingredients

In one embodiment, the TPU in the embodiment 1 may optionally comprise or runner ingredients, for example catalysts and additives.

Suitable additives are known per se to the person skilled in the art. In one embodiment, the TPU in the embodiment 1 further comprises of additives selected from surface-active substances, flame retardants, nucleating agents, oxidation stabilizers, antioxidants, lubricants and demolding aids, dyes and pigments, stabilizers, for example, against hydrolysis, light, heat or discoloration, inorganic and/or organic fillers, reinforcers and plasticizers. Suitable additives can be found, for example, in the Kunststoffhandbuch, volume VII, published by Vieweg and Höchtlen, Carl Hanser Verlag, Munich 1966 (p. 103-113).

Suitable catalysts are likewise known in the prior art. These include, such as but not limited to, organic metal compounds selected from tin, titanium, zirconium, hafnium, bismuth, zinc, aluminum and iron organyls, for example tin organyl compounds, such as dialkyls, for example, tin(II) isooctoate, tin dioctoate, dimethyltin or diethyltin, or tin organyl compounds of aliphatic carboxylic acids, such as tin diacetate, tin dilaurate, dibutyltin diacetate, dibutyltin dilaurate, titanate esters, bismuth compounds, such as bismuth alkyl compounds, such as bismuth neodecanoate or similar, or iron compounds, such as iron(III) acetylacetonate.

In one embodiment, further ingredients are present in the TPU in the embodiment 1 in an amount in between 0.1 wt. % to 10 wt. %, based on the total weight of the TPU.

Medical Tubing

The viscoelastic TPU material in the embodiment 1, due to its advantageous properties, finds application in medical tubing. In the present context, medical tubing can be selected from insulin infusion tubing, catheter for blood transport, dialysis tubing, enteral feeding system, oxygen tubing, drainage tubing, peristaltic pump tubing, central venous catheters, peripherally inserted central catheters, arterial lines, ports, renal infusion systems, drainage catheters and haemodialysis catheters.

In one embodiment, the medical tubing in the embodiment 1 is selected from insulin infusion tubing, catheter for blood transport, dialysis tubing, enteral feeding system, oxygen tubing, drainage tubing, peristaltic pump tubing, central venous catheters, peripherally inserted central catheters, arterial lines, ports, and renal infusion systems.

In another embodiment, the medical tubing in the embodiment 1 is selected from insulin infusion tubing, catheter for blood transport, dialysis tubing, enteral feeding system, oxygen tubing, drainage tubing, peristaltic pump tubing, and central venous catheters. In still another embodiment, it is selected from insulin infusion tubing, catheter for blood transport, and dialysis tubing.

In yet another embodiment, the medical tubing in the embodiment 1 is insulin infusion tubing.

In one embodiment, the medical tubing in the embodiment 1 comprises at least one layer made from the TPU material, as described herein. It is also possible that the medical tubing in the embodiment 1 comprises of more than one layer, for example a multi-layered structure. In the present context, the term “multilayer” refers to the presence of at least 2 layers in the medical tubing. In one embodiment, the multilayer structure can comprise more than 2 layers, for example, 3, 4, 5, 6 or 7 layers. Such layers can be referred to as intermediate layers. It is further possible that one or more of these layers are made from the TPU material, as described herein, while the other layers are made from other plastic materials, for example, PVC.

In one embodiment, the medical tubing in the embodiment 1 has an opening at each end thereof. Suitable diameter of the opening depends on the specific use of these tubings and are therefore, well known to the person skilled in the art. In another embodiment, the medical tubing in the embodiment 1 comprises walls that have a thickness in the range of 0.2 mm to 2 mm.

In yet another embodiment, the medical tubing in the embodiment 1 has a cylindrical or non-cylindrical shape. These shapes are generally manufactured by using techniques such as, but not limited to, co-extruding of the TPU material, as described herein.

Advantageously, the medical tubing in the embodiment 1 comprises or the transparent TPU material having the required shore hardness, Tg and hard segment content, which provides for acceptable mechanical properties, such as but not limited to, tensile strength, elongation at break, modulus and tear strength.

Process

Another aspect of the present invention is embodiment 2, directed to a process for preparing the medical tubing of the embodiment 1, said process comprising at least the step of extruding the TPU obtained by reacting:

(a) polyisocyanate composition,

(b) at least one chain extender, and

(c) at least one polyol composition,

wherein the polyol composition comprises at least one polyester polyol (P1) which has a weight average molecular weight Mw in the range of 500 g/mol to 3000 g/mol and has at least one aromatic polyester block (B1), wherein the aromatic polyester block (B1) is present in between 20 wt. % to 80 wt. %, based on the total weight of the polyester polyol (P1), wherein the TPU has a shore hardness of less than 95 A, determined according to DIN ISO 7619.

In one embodiment, suitable further ingredients can be added in the embodiment 2. It is also to be understood that the reaction between (a) polyisocyanate composition, (b) at least one chain extender, and (c) at least one polyol composition, can be conducted under the conditions that are known per se. The reaction may be conducted in the presence of further ingredients, if required. In another embodiment, the TPU in the embodiment 1 or 2 is obtained by conducting the reaction at higher temperatures than room temperature, for example, in the range between 50° C. to 250° C., or in between 50° C. to 200° C. In yet another embodiment, the temperature is in the range of 50° C. to 150° C., or in between 60° C. to 120° C. According to the invention, heating can be effected in any suitable manner known to the person skilled in the art, for instance, by electrical heating, heating via heated oil, heated polymer fluids or water, induction fields and hot air or IR irradiation.

Illustrative embodiments of the present invention are listed below, but do not restrict the present invention. In particular, the present invention also encompasses those embodiments that result from the dependency references and hence combinations specified hereinafter. More particularly, in the case of naming of a range of embodiments hereinafter, for example the expression “The process according to any of embodiments 1 to 4”, should be understood such that any combination of the embodiments within this range is explicitly disclosed to the person skilled in the art, meaning that the expression should be regarded as being synonymous to “The process according to any of embodiments 1, 2, 3 and 4”:

I. A medical tubing comprising a thermoplastic polyurethane obtained by reacting:

(a) a polyisocyanate composition,

(b) at least one chain extender, and

(c) at least one polyol composition,

wherein the polyol composition comprises at least one polyester polyol (P1) which has a weight average molecular weight Mw in the range of 500 g/mol to 3000 g/mol and has at least one aromatic polyester block (B1), wherein the aromatic polyester block (B1) is present in between 20 wt. % to 80 wt. % based on the total weight of the polyester polyol (P1),

wherein the thermoplastic polyurethane has a shore hardness of less than equal to 95 A, determined according to DIN ISO 7619.

II. The medical tubing according to embodiment I, wherein a molar ratio between the at least one chain extender (b) and the at least one polyester polyol (P1) is in between 1.0:5.0 to 5.0:1.0.
III. The medical tubing according to embodiment II, wherein the molar ratio is in between 1.0:1.0 to 5.0:1.0.
IV. The medical tubing according to one or more of embodiments I to III, wherein the thermoplastic polyurethane has a shore A hardness ranging between 30 to 95 determined according to DIN ISO 7619.
V. The medical tubing according to one or more of embodiments I to IV, wherein the thermoplastic polyurethane has a shore A hardness ranging between 50 to 95 determined according to DIN ISO 7619.
VI. The medical tubing according to one or more of embodiments I to V, wherein the thermoplastic polyurethane has a shore A hardness ranging between 70 to 95 determined according to DIN ISO 7619.
VII. The medical tubing according to one or more of embodiments I to VI, wherein the thermoplastic polyurethane has a glass transition temperature Tg ranging between −5° C. to 30° C., determined by dynamic mechanical analysis according to DIN EN ISO 6721 from −80° C. to 140° C. in increments of 5° C. at a frequency of 1 Hz in torsion mode.
VIII. The medical tubing according to one or more of embodiments I to VII, wherein the hard segment content in the thermoplastic polyurethane is less than 50%, as determined according to the description.
IX. The medical tubing according to one or more of embodiments I to VIII, wherein the hard segment content in the thermoplastic polyurethane is in between 10% to 45%, as determined according to the description.
X. The medical tubing according to one or more of embodiments I to IX, wherein the hard segment content in the thermoplastic polyurethane is in between 20% to 45%, as determined according to the description.
XI. The medical tubing according to one or more of embodiments I to X, wherein the at least one aromatic polyester block (B1) is present in between 20 wt. % to 70 wt. %, based on the total weight of the polyester polyol (P1).
XII. The medical tubing according to one or more of embodiments I to XI, wherein the at least one aromatic polyester block (B1) is a polyester of an aromatic dicarboxylic acid and an aliphatic diol.
XIII. The medical tubing according to one or more of embodiments I to XII, wherein the at least one aromatic polyester block (B1) is a polyethylene terephthalate block or a polybutylene terephthalate block.
XIV. The medical tubing according to one or more of embodiments I to XIII, wherein the at least one chain extender has a molecular weight in between 60 g/mol to 300 g/mol.
XV. The medical tubing according to one or more of embodiments I to XIV, wherein the polyisocyanate composition comprises an aliphatic polyisocyanate or an aromatic polyisocyanate or a mixture thereof.
XVI. The medical tubing according to one or more of embodiments I to XV, wherein the polyisocyanate composition comprises an aromatic polyisocyanate.
XVII. The medical tubing according to embodiments XVI, wherein the aromatic polyisocyanate is selected from toluene diisocyanate; polymeric toluene diisocyanate, methylene diphenyl diisocyanate and/or polymeric methylene diphenyl diisocyanate; m-phenylene diisocyanate; 1,5-naphthalene diisocyanate; 4-chloro-1; 3-phenylene diisocyanate; 2,4,6-toluylene triisocyanate, 1,3-diisopropylphenylene-2,4-diisocyanate; 1-methyl-3,5-diethylphenylene-2,4-diisocyanate; 1,3,5-triethylphenylene-2,4-diisocyanate; 1,3,5-triisoproply-phenylene-2,4-diisocyanate; 3,3′-diethyl-bisphenyl-4,4′-diisocyanate; 3,5,3′,5′-tetraethyl-diphenylmethane-4,4′-diisocyanate; 3,5,3′,5′-tetraisopropyldiphenylmethane-4,4′-diisocyanate; 1-ethyl-4-ethoxy-phenyl-2,5-diisocyanate; 1,3,5-triethyl benzene-2,4,6-triisocyanate; 1-ethyl-3,5-diisopropyl ben-zene-2,4,6-triisocyanate, tolidine diisocyanate, and 1,3,5-triisopropyl benzene-2,4,6-triisocyanate.
XVIII. The medical tubing according to embodiment XVI or XVII, wherein the aromatic polyisocyanate is methylene diphenyl diisocyanate and/or polymeric methylene diphenyl diisocyanate.
XIX. The medical tubing according to one or more of embodiments I to XVIII, wherein the thermoplastic polyurethane further comprises at least one cross-linking agent.
XX. The medical tubing according to embodiment XIX, wherein the at least one cross-linking agent comprises a thermoplastic polyurethane carrier and an isocyanate component.
XXI. The medical tubing according to embodiment XIX or XX, wherein the at least one crosslinking agent is present in an amount in between 0.1 wt. % to 10 wt. %, based on the total weight of the thermoplastic polyurethane.
XXII. The medical tubing according to one or more of embodiments I to XXI, wherein the thermoplastic polyurethane further comprises additives.
XXIII. The medical tubing according to one or more of embodiments I to XXII, wherein the medical tubing is selected from insulin infusion tubing, catheter for blood transport, dialysis tubing, enteral feeding system, oxygen tubing, drainage tubing, peristaltic pump tubing, central venous catheters, peripherally inserted central catheters, arterial lines, ports, renal infusion systems, drainage catheters and haemodialysis catheters.
XXIV. The medical tubing according to one or more of embodiments I to XXIII, wherein the medical tubing comprises walls that have a thickness in the range from 0.2 to 2 mm.
XXV. A process for preparing a medical tubing according to one or more of embodiments I to XXIV, said process comprising at least the step of extruding the thermoplastic polyurethane obtained by reacting:

(a) a polyisocyanate composition,

(b) at least one chain extender, and

(c) at least one polyol composition,

wherein the polyol composition comprises at least one polyester polyol (P1) which has a weight average molecular weight Mw in the range of 500 to 3000 g/mol and has at least one aromatic polyester block (B1), wherein the aromatic polyester block (B1) is present in between 20 wt. % to 80 wt. % based on the total weight of the polyester polyol (P1), and

wherein the thermoplastic polyurethane has a shore hardness of less than equal to 95 A determined according to DIN ISO 7619.

XXVI. A medical tubing comprising a thermoplastic polyurethane obtained by reacting:
a polyisocyanate composition comprising (i) MDI and/or polymeric MDI or (ii) aliphatic isocyanate,
at least one chain extender, and
at least one polyol composition,
   wherein the polyol composition comprises at least one polyester polyol (P1) which has a weigh average molecular weight Mw in the range of 500 g/mol to 3000 g/mol and has at least one aromatic polyester block (B1), wherein the aromatic polyester block (B1) is present in between 20 wt. % to 80 wt. % based on the total weight of the polyester polyol (P1),
   wherein the thermoplastic polyurethane has a shore hardness of less than equal to 95 A, determined according to DIN ISO 7619.
   XXVII. A medical tubing comprising a thermoplastic polyurethane obtained by reacting:
a polyisocyanate composition containing (i) MDI and/or polymeric MDI or (ii) aliphatic isocyanate,
at least one chain extender, and
at least one polyol composition,
   wherein the polyol composition comprises at least one polyester polyol (P1) which has a weigh average molecular weight Mw in the range of 500 g/mol to 3000 g/mol and has at least one aromatic polyester block (B1), wherein the aromatic polyester block (B1) is present in between 20 wt. % to 80 wt. % based on the total weight of the polyester polyol (P1),
   wherein the thermoplastic polyurethane has a shore hardness of less than equal to 95 A, determined according to DIN ISO 7619.

### EXAMPLES

The presently claimed invention is illustrated by the non-restrictive examples which are as follows:

### Feedstock

| | |
|---|---|
| PESOL | Polyester polyol based on adipic acid, PET and |
| | diethylene glycol, with hydroxyl number of 53.4 mg |
| | KOH/g (Mw of 2200 g/mol), functionality: 2.0 |
| Iso | Monomeric MDI (4,4′-MDI) |
| Chain extender | CE1: butane-1,4-diol |
| | CE2: hexane-1,6-diol |
| Additive | AD1: Sterically hindered primary phenolic |
| | antioxidant stabilizer |
| Cross-linking agent | CL1: Elastollan ® X-Flex from BASF |

### Standard Methods

| | |
|---|---|
| Density | DIN EN ISO 1183-1A |
| Shore hardness | DIN ISO 7619 |
| Tensile strength | DIN 53504 |
| Elongation at break | DIN 53504 |
| Glass transition temperature, Tg | DIN EN ISO 6721 |
| Hydroxyl number | DIN 53240 |
| Acid number | DIN EN 1241 |

### Determination of Viscosity

Unless stated otherwise, the viscosity of PESOL was determined at 75° C. by DIN EN ISO 3219 (Jan. 10, 1994 edition) with a Rheotec RC 20 rotary viscometer using the CC 25 DIN spindle (spindle diameter: 12.5 mm; internal measuring cylinder diameter: 13.56 mm) at a shear rate of 50 l/s.

Synthesis of Polyester Polyol with PET Blocks (PESOL)

A 4000 ml round-neck flask provided with PT100 thermocouple, nitrogen inlet, stirrer, column, column head, Anschütz-Thiele attachment and heating mantle were initially charged with 1099.59 g of adipic acid and 921.43 g of diethylene glycol (no excess). The mixture was then heated to 120° C. until a homogeneous mixture is formed. 750 g of polyethylene terephthalate (PET) were then added to the mixture in the form of flakes, and then 10 ppm=2.5 g of TTB (tetra-n-butyl orthotitanate, 1% in toluene). The reaction mixture was heated first to 180° C. for about 1.5 h and then further to 240° C., and the resultant water of reaction was continuously removed. Over the entire synthesis, the PET flakes were gradually degraded, and a transparent mixture was formed, which as condensed until a product having an acid number<1.0 mg KOH/g was obtained. The PESOL had the following properties:

Hydroxyl number: 53.4 mg KOH/g;
Acid number: 0.38 mg KOH/g
Viscosity at 75° C.: 1936 mPas

### General Synthesis of TPU

The PESOL was initially charged in a container at 80° C. and mixed by vigorous stirring with the components listed in Table 1. The reaction mixture was heated to above 110° C. and was then poured out onto a heated, teflon-coated table. The cast slab obtained was heat-treated at 80° C. for 15 h, then pelletized and processed.

TABLE 1
Example compounds used

| | I.E. 1 | I.E. 2 | I.E. 3 | C.E. 1 | I.E. 4 |
|---|---|---|---|---|---|
| Ingredient | (wt. %) | (wt. %) | (wt. %) | (wt. %) | (wt. %) |
| PESOL | 57.79 | 62.03 | 53.61 | 44.68 | 65.03 |
| Iso | 32.55 | 27.44 | 35.79 | 42.08 | 26.50 |
| CE1 | 9.20 | — | 10.59 | 13.24 | 6.49 |
| CE2 | 0.46 | 9.53 | — | — | — |
| AD1 | — | — | — | — | 1.00 |
| CL1 | — | — | — | — | 1.00 |
| Molar ratio | 3.77:1.0 | 2.77:1.0 | 4.52:1.0 | 6.84:1.0 | 2.27:1.0 |
| (CE:PESOL) | | | | | |
| Hard segment | 35% | 30% | 40% | 50% | 24.50% |
| Content | | | | | |

### Mechanical Properties

The measurements collated in Table 2 were established using 2.0 mm injection molded plaques, cut to dimensions as described in the standards above.

TABLE 2
Mechanical properties of inventive and comparative examples

| | | Elongation | Tensile | | |
|---|---|---|---|---|---|
| | Shore | at break | strength | Tg (° C.) | |
| Examples | hardness | (%) | (MPa) | G″ | Tan delta |
| I.E. 1 | 92A | 540 | 54 | −5 | 10 |
| I.E. 2 | 83A | 640 | 46 | −5 | 0.5 |
| I.E. 3 | 93A | 346.36 | 32.34 | 16 | 36 |
| C.E. 1 | 98A | 5.58 | 49.20 | 31 | 44 |
| I.E. 4 | 87A | 471.90 | 42.78 | 3 | 14 |

As evident in Table 2 above, the inventive examples 1 to 4 have substantially improved elongation properties and comparable tensile strength values than those of the comparative ones. Further, the shore hardness value, hard segment content and Tg values of the inventive examples are within the range as required for medical tubing application.

### Tube Extrusion

The process parameters under which I.E. 1 was compounded is set forth in Table 3 below. The ingredients were fed into the single screw extruder in a first zone (Zone 1) and passed through a series of additional zones (Zones 2-7) that were heated to varying temperatures. Then, the TPU composition was pushed through a tube die to form the strands which were cooled with water and pelletized. Tubings were finally obtained with an outer diameter of 6.35 mm and in inner diameter of 5.33 mm.

TABLE 3
Extrusion process parameters

| | |
|---|---|
| Zone 1 temperature (° C.) | 190 |
| Zone 2 temperature (° C.) | 210 |
| Zone 3 temperature (° C.) | 215 |
| Gate (° C.) | 225 |
| Adapter (° C.) | 230 |
| Die temperature (° C.) | 210 |
| Pressure (bar) | 250 |
| Screw speed (RPM) | 20 |
| Screw ratio | 1:2.5 |
| Bulk temperature | 215 |

1: A medical tubing, comprising a thermoplastic polyurethane obtained by reacting:
a polyisocyanate composition,
at least one chain extender, and
at least one polyol composition,
wherein the at least one polyol composition comprises at least one polyester polyol (P1) which has a weight average molecular weight Mw in a range of 500 g/mol to 3000 g/mol and has at least one aromatic polyester block (B1), wherein the at least one aromatic polyester block (B1) is present in an amount between 20 wt. % to 80 wt. % based on a total weight of the at least one polyester polyol (P1),
wherein the thermoplastic polyurethane has a shore hardness of less than or equal to 95 A, determined according to DIN ISO 7619.

2: The medical tubing according to claim 1, wherein a malar ratio between the at least one chain extender and the at least one polyester polyol (P1) is in between 1.0:5.0 to 5.0:1.0.

3: The medical tubing according to claim 2, wherein the molar ratio is in between 1.0:1.0 to 5.0:1.0.

4: The medical tubing according to claim 1, wherein the thermoplastic polyurethane has a shore A hardness ranging between 30 to 95, determined according to DIN ISO 7619.

5: The medical tubing according to claim 1, wherein the thermoplastic polyurethane has a glass transition temperature Tg ranging between −5° C. to 30° C., determined by dynamic mechanical analysis according to DIN EN ISO 6721 from −80° C. to 140° C. in increments of 5° C. at a frequency of 1 Hz in torsion mode.

6: The medical tubing according to claim 1, wherein a hard segment content in the thermoplastic polyurethane is less than 50%,
wherein the hard segment content is defined by the formula:
$$Hard segment fraction = \{ {\sum }_{x = 1}^{k} [ ( {m}_{KV , CE} / {M}_{KV , CE} ) * {M}_{Iso} + {m}_{KV , CE} ] \} / {m}_{total}$$
wherein
m_{KV,CE} is a mass of the at least one chain extender in g,
M_{KV,CE} is a mole mass of the at least one chain extender in g/mol,
M_{Iso} is a molar mass of the polyisocyanate in g/mol,
mₜₒₜₐₗ is a total mass of all starting materials in g, and
k is a number of chain extenders.

7: The medical tubing according to claim 6, wherein the hard segment content in the thermoplastic polyurethane is in between 10% to 45%.

8: The medical tubing according to claim 1, wherein the at least one aromatic polyester block (B1) is a polyester of an aromatic dicarboxylic acid and an aliphatic diol.

9: The medical tubing according to claim 1, wherein the at least one aromatic polyester block (B1) is a polyethylene terephthalate block or a polybutylene terephthalate block.

10: The medical tubing according to claim 1, wherein the thermoplastic polyurethane further comprises at least one crosslinking agent.

11: The medical tubing according to claim 10, wherein the at least one cross-linking agent comprises a thermoplastic polyurethane carrier and an isocyanate component.

12: The medical tubing according to claim 1, wherein the thermoplastic polyurethane further comprises additives.

13: The medical tubing according to claim 1, wherein the medical tubing is selected from the group consisting of an insulin infusion tubing, a catheter for blood transport, a dialysis tubing, an enteral feeding system, an oxygen tubing, a drainage tubing, a peristaltic pump tubing, a central venous catheter, a peripherally inserted central catheter, an arterial a port, a renal infusion system, a drainage catheter, and a hemodialysis catheter.

14: The medical tubing according to claim 1, wherein the medical tubing comprises walls that have a thickness in a range from 0.2 to 2 mm.

15: A process for preparing the medical tubing according to claim 1, said process at least comprising:
extruding the thermoplastic polyurethane.
